(19) United States
(12) Patent Application Publication
(10) Pub. No.: US 2019/0032013 A1
(43) Pub. Date: Jan. 31, 2019

(54) IMMUNE CELL SELECTION, EXPANSION, AND USE

(71) Applicant: TRACT THERAPEUTICS, INC., Chicago, IL (US)

(72) Inventor: Joseph R. LEVENTHAL, Chicago, IL (US)

(21) Appl. No.: 16/072,345

(22) PCT Filed: Jan. 27, 2017
(86) PCT No.: PCT/US17/15238

§ 371 Date: Jul. 24, 2018

Related U.S. Application Data
(60) Provisional application No. 62/288,574, filed on Jan. 29, 2016.

(51) Int. Cl. C12N 5/0783; A61P 37/06; A61K 39/00

(52) U.S. Cl. CPC C12N 5/0637; A61P 37/06; A61K 39/0008; C12N 2501/999; C12N 2501/2302; C12N 2501/15; A61K 39/001

(57) ABSTRACT

Methods, compositions, and kits for generating therapeutically relevant populations immunosuppressive T-reg cells and uses thereof are disclosed.

### RELATED APPLICATION

This application claims the benefit of U.S. Provisional Patent Application No. 62/288,574 filed Jan. 29, 2016, which is incorporated by reference herein in its entirety.

### FIELD

The disclosure relates generally to the selection, expansion, and use of T-reg cell populations.

### BACKGROUND

Significant limitations in the efficacy of organ and tissue transplant include the rejection of allografts by host immune systems and graft versus host disease. Pharmaceutical immunosuppressants are commonly used to treat these conditions; however, they are not always effective. Regulatory T cells (T-reg cells) are potent suppressor regulatory T lymphocytes (CD4+/CD25+) that have been demonstrated to have importance in the active immune regulation/suppression in the processes of graft rejection and tolerance. Current methodologies for generating therapeutically relevant numbers of T-reg cells rely on purification and ex vivo expansion of freshly isolated T-reg cells. However, the logistics of these methodologies, including the requirement of freshly isolated T-reg cells, the relatively low numbers of expanded T-reg cells, as well as the time required for expansion present considerable disadvantages. Therefore, there is a need for new approaches for providing T-reg cells for therapeutic purposes that overcome these disadvantages.

### SUMMARY

In a first aspect, the present disclosure provides a method for selecting and expanding a population of CD4+/CD25+ T-regulatory cells including thawing a frozen apheresis sample received from an individual, selecting a population of CD4+/CD25+ T-regulatory (T-reg) cells from the thawed apheresis sample, and culturing the selected population of CD4+/CD25+ T-reg cells to produce an expanded population of CD4+/CD25+ T-reg cells. The expanded population of CD4+/CD25+ T-reg cells may be larger than the selected population of CD4+/CD25+ T-reg cells by a factor of about 40.

In one embodiment, a percentage of CD4+ T-reg cells in the expanded population of CD4+/CD25+ T-reg cells differs from a percentage of CD4+ cells in an expanded population of CD4+/CD25+ T-reg cells selected from a fresh, non-frozen apheresis product by less than about 3%. In another embodiment, a percentage of CD25+ T-reg cells in the expanded population of CD4+/CD25+ T-reg cells differs from a percentage of CD25+ cells in an expanded population of CD4+/CD25+ T-reg cells selected from a fresh, non-frozen apheresis product by less than about 5%.

In a second aspect, the present disclosure provides a method for enriching and expanding CD4+/CD25+ T-regulatory (T-reg) cells from a cryopreserved apheresis sample including thawing the apheresis sample, suspending the thawed sample in a buffer comprising Human Serum Albumin (HSA), Magnesium Chloride (MgCl₂), and Dornase alfa, selecting a population of CD4+/CD25+ T-regulatory (T-reg) cells from the suspended apheresis sample to produce a selected population, and culturing the selected population of CD4+/CD25+ T-reg cells to produce an expanded population of CD4+/CD25+ T-reg cells. The expanded population of CD4+/CD25+ T-reg cells may be larger than the selected population of CD4+/CD25+ T-reg cells by a factor of about 30.

In a third aspect, the present disclosure provides a method for selecting CD25+ T-regulatory cells including thawing a cryopreserved apheresis product comprising T-cells, washing the thawed product in a buffer comprising Human Serum Albumin (HSA), Magnesium Chloride (MgCl₂), and Dornase alfa, incubating the thawed product with one or more capture surfaces comprising a binding agent for CD8+ and CD19+ cells, capturing the CD8+/CD19+ cells to the one or more surfaces, collecting a CD8/CD19 depleted product by washing the one or more surfaces with the buffer, and combing the CD8/CD19 depleted product with a capture surface for CD25+ cells, eluting cells captured on the capture surface for CD25+ cells with the buffer to provide a CD25+ enriched product. One or more of steps (c) though (g) use one or more buffers comprising HSA, MgCl₂, and Dornase alfa.

In a fourth aspect, the present disclosure provides a method for expanding a CD25+ cell population including culturing CD25+ cells in a growth media supplemented with Interleukin-2 (IL-2), rapamycin, and Transforming Growth Factor Beta (TGF-β) in the presence of one or more surfaces comprising an anti-CD3+ antibody and anti-CD28+ antibody for about two days, adding IL-2 to the growth media and culturing the cells for about three days, adding additional growth media and IL-2, rapamycin, and TGF-β and culturing the cells for about two days, adding additional growth media and IL-2, rapamycin, TGF-β, and one or more surfaces comprising an anti-CD3+ antibody and anti-CD28+ antibody and culturing the cells for about two days, adding IL-2, rapamycin, and TGF-β, and culturing the cells for about 3 days, adding IL-2 and culturing the cells for about 2 days, adding additional growth media, IL-2, and TGF-β, and culturing the cells for about three days, adding IL-2 and culturing the cells for about two days, and separating the cell culture from the one or more capture surfaces to provide an expanded CD25+ cell population. In one embodiment, no additional rapamycin is added to the cells beyond about 9 days of culture.

In a fifth aspect, the present disclosure provides a kit for providing an expanded and enriched CD4+/CD25+ T-reg cell population including a buffer comprising HSA, MgCl₂, and Dornase alfa and instructions for use.

In a sixth aspect, the present disclosure provides a kit for suppressing the immune system of an individual in need thereof including an expanded and enriched CD4+/CD25+ T-reg cell population and instructions for use.

In an eighth aspect, the present disclosure provides a composition comprising a previously frozen apheresis sample, a buffer comprising HSA, MgCl₂, and Dornase alfa.

In a ninth aspect, the present disclosure provides a method for treating an organ transplant recipient including administering to the recipient between about 1,000,000,000 and 5,000,000,000 CD4+/CD25+ T-reg cells selected and expanded from a frozen apheresis product.

In one embodiment, the frozen apheresis product was taken from the recipient prior to organ transplant. In another embodiment, the frozen apheresis product was taken from a donor that is not the recipient. In a further embodiment, the method at least one of reduces, stops, and prevents a cellular immune response that causes cellular, organ, or tissue rejection in the recipient.

In a tenth aspect, the present disclosure provides a method for treating an organ transplant recipient including administering to the patient between about 1,000,000,000 and 5,000,000,000 CD4+/CD25+ T-reg cells from a population of cells prepared by culturing CD25+ cells in a growth media supplemented with Interleukin-2 (IL-2), rapamycin, and Transforming Growth Factor Beta (TGF-β) in the presence of one or more surfaces comprising an anti-CD3+ antibody and anti-CD28+ antibody for about two days, adding IL-2 to the growth media and culturing the cells for about three days, adding additional growth media and IL-2, rapamycin, and TGF-β and culturing the cells for about two days, adding additional growth media and IL-2, rapamycin, TGF-β, and one or more surfaces comprising an anti-CD3+ antibody and anti-CD28+ antibody and culturing the cells for about two days, adding IL-2, rapamycin, and TGF-β, and culturing the cells for about 3 days, adding IL-2 and culturing the cells for about 2 days, adding additional growth media, IL-2, and TGF-β, and culturing the cells for about three days, adding IL-2 and culturing the cells for about two days, and separating the cell culture from the one or more capture surfaces to provide an expanded CD25+ cell population. No additional rapamycin may be added to the cells beyond 9 days of culture.

In an eleventh aspect, the present disclosure provides a composition including a phosphate-buffered saline supplemented with 1 mM EDTA, 5% human serum albumin, 3.5 mM MgCl₂, and 50 U/mL Dornase alfa.

Other aspects, embodiments, and implementations will become apparent to those of ordinary skill in the art by reading the following detailed description, with reference where appropriate to the accompanying drawings.

### DESCRIPTION OF THE FIGURES

The following detailed description of the embodiments of the present disclosure can be best understood when read in conjunction with the following figures, in which:

FIG. 1 illustrates phenotypic characterization of cell populations during the two-column cell selection process using the CliniMACS® Magnetic Column device, as described in Example No. 1 and Table No. 2. Data shown indicate percentages of CD20+, CD8+, CD4+, CD25+, and FoxP3+ cells before selection (Apheresis), for the population of cells removed (Neg Sel), and for the population selected (Day 0/Pos Sel);

FIG. 2 shows a schematic of a protocol for large-scale expansion of CD4+/CD25+ T-reg cells;

FIG. 3 shows the ‘growth kinetics’ of fresh versus frozen cells. Apheresis products from a normal donor were split into two samples, where one sample was used fresh and the other sample was cryopresserved before cellular subpopulation selection followed by T-reg cell culture expansion. The cryopreserved sample was thawed using a thawing buffer including a DNAse containing PBS/EDTA buffer and then used for cellular subpopulation selection followed by T-reg cell culture expansion. Cell population growth was measured at culture initiation (Day 0) during the growth process (Day 14), and at the end of the process upon reaching the final product (Day 21);

FIG. 4 shows enriched and expanded CD4+/CD25+ T-reg cells that were generated from fresh or cryopreserved apheresis products and evaluated for their functional ability to induce suppression in a MLR assay. At T-reg:T responder cell ratios of 1:2 through 1:32 there was no difference in the immunosuppressive function of T-reg cells generated from fresh or frozen apheresis products; and

FIG. 5 shows that T-reg cells expanded for 21 days from peripheral blood lymphocytes of renal failure patients had acceptable immune suppressive function at several T-reg:T responder cell ratios.

FIG. 6, Panel A shows a schematic protocol for the expansion of T-reg cells: CD4+/CD25+ cells purified from leukapheresis product from renal transplant recipients were stimulated with anti-CD3-CD28 beads (MACS ExpBeads) in the presence of IL-2, TGFβ, and Sirolimus as indicated. FIG. 6, Panel B is a representation of the clinical protocol for the use of T-Cell therapy for kidney transplant recipients.

FIG. 7A shows growth curves of T-reg cells (absolute number) in nine expansion cultures.

FIG. 7B shows flow cytometric analyses of T-regs on days 0, 14 and 21 of the culture.

FIG. 7C shows the phenotype of T-reg cells in an expansion culture.

FIG. 8, Panels 8A, 8B, 8C and 8D, shows the profile of T-reg cell surface receptor expressions. On the left 2 columns are representative data on the relative intensities of indicated receptors on freshly isolated T-regs on day 0 (grey) versus day 21 (black), and on the right 2 columns are their mean fluorescent intensities (MFI) in all 9 expansion cultures.

FIG. 9 shows the percent clonality in a T cell Receptor (TCR) clonal repertoire determined from DNA taken from aliquots of apheresis products and 21-day expanded T-regs from indicated recipients (patient samples 1-6) that were subjected to high-throughput sequencing (ImmunoSEQ analysis by Adaptive Biotechnologies).

FIGS. 10A and 10B show the results of an experiment to measure the immunoregulatory capabilities of expanded T-regs. FIGS. 10A the counts per minute (CPM) values with the various modulators at indicated modulator: T responder ratios. FIG. 10B shows a mean±SD percentage of suppression that were calculated for each individual experiment (n=9).

FIGS. 10C and 10D shows the results of an experiment to show the immunoregulatory capabilities of expanded T-regs. In the portion of the experiment shows in FIG. 10C, the CD4 cells that were negative for PKH26 and CD127 and then those that diluted the CFSE were sequentially gated and analyzed for CD25 and FOXP3 expressions. Thus the cells of interest were CD4⁺CD127⁻CD25⁻FOXP3⁺ T-regs in the CFSE diluted proliferating responders. A representative experiment with “donor” and third party stimulators at 1:8 modulator: T responder ratio is shown. FIG. 10D shows the percentages of T-regs obtained with the T-reg modulators that were divided by those obtained with the Rx control modulators to calculate the fold change. Thus, in the example in FIG. 10C right, the fold change was 85.7÷40.1=2.13. This fold change in T-regs was plotted for indicated T-reg: T responder ratios for each experiment. The data are shown as mean±SD fold change from expansion of nine samples (*** p<0.001).

FIGS. 11A and 11B show the results of immune monitoring in blood of T-reg recepicients. FIG. 11A shows flow cytometric analyses performed using whole blood, and the absolute number of indicated subsets were serially monitored. FIG. 11B shows the percent of Foxp3⁺ T-regs observed in the recipient PBMC at pre-transplant was considered as 1, and the fold change in relation to that was calculated during the post-transplant period for each patient.

### DESCRIPTION

All publications, patents and patent applications cited herein are hereby expressly incorporated by reference for all purposes.

Before describing the present invention in detail, a number of terms will be defined. As used herein, the singular forms “a,” “an,” and “the” include plural referents unless the context clearly dictates otherwise. For example, reference to a “nucleic acid” means one or more nucleic acids.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

As used herein, the term “about” refers to ±10% of any particular value unless otherwise noted.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z”.

As used herein, the term “viability” when used to describe a cell population, refers to the percentage of viable cells within the population.

As used herein, the term “therapeutically relevant” in the context of administration of T-reg cells to a recipient in need thereof, refers to the number of T-reg cells that can be administered to the recipient to cause an ameliorating effect to the recipient. In one embodiment, a therapeutically relevant number of T-reg cells is any number of T-reg cells that at least one of reduces, stops, and/or prevents a cellular immune response that causes cellular, organ, or tissue rejection. In one particular embodiment, a therapeutically relevant number of T-reg cells is about 1×10⁹ to about 5×10⁹ T-reg cells administered at one time, post organ transplant to a lymphodepleted transplant recipient. The T-reg cells can be administered administered at approximately 45-75 days post organ transplant, for instance, at 45, 50, 55, 60, 65, 70 or 75 days post transplant.

The term “autoimmune disease” as used herein is defined as a disorder that results from an autoimmune response. An autoimmune disease is the result of an inappropriate and excessive response to a self-antigen. Examples of autoimmune diseases include, but are not limited to, Addision's disease, alopecia areata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

For the purposes of describing and defining the present disclosure, it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Disclosed herein are compositions, methods, and kits for producing sterile, cellular therapy products that may be specifically created from and/or for an individual, for example, for suppressing the individual's immune system by administering the product to the individual. In one embodiment, a cellular therapy product intended for an individual is derived from an apheresis product taken from that individual. In another embodiment, a cellular therapy product intended for an individual is derived from an apheresis product taken from another individual or from another cell source.

In one embodiment, a contemplated cellular therapy product includes an enriched and expanded CD4+/CD25+ T-regulatory (T-reg) cell population. For example, the population may include greater than about 80% CD4+ cells, or greater than about 85% CD4+ cells, or greater than about 90% CD4+ cells, or greater than about 95% CD4+ cells, or greater than about 98% CD4+ cells. Further, the population may include greater than about 80% CD25+ cells, or greater than about 85% CD25+ cells, or greater than about 90% CD25+ cells, or greater than about 95% CD25+ cells, or greater than about 98% CD25+ cells. Further, the population may include greater than about 20% FoxP3+ cells, or greater than about 25% FoxP3+ cells, or greater than about 30% FoxP3+ cells, or greater than about 35% FoxP3+ cells, or greater than about 40% FoxP3+ cells.

Further, the enriched and expanded CD4+/CD25+ T-reg cell population may have diminished amounts or may be devoid of other cells exhibiting specific antigens. For example, the population may include less than about 10% CD8+ cells, or less than about 5% CD8+ cells, or less than about 3% CD8+ cells, or less than about 2% CD8+ cells, or less than about 1% CD8+ cells. Similarly, the population may include less than about 10% CD20+ cells, or less than about 5% CD20+ cells, or less than about 3% CD20+ cells, or less than about 2% CD20+ cells, or less than about 1% CD20+ cells. Further, it should be noted that both CD19 and CD20 can be used as markers for B cells, and the use of either or both for phenotyping and/or targeting for depletion of B cells from T-reg cell populations is contemplated herein.

It is further contemplated that the enriched and expanded CD4+/CD25+ T-reg cell population may have greater than about 80% viability, or greater than about 90% viability, or greater than about 95% viability, or greater than about 98% viability.

In another embodiment, the apheresis products contemplated herein may be obtained from an individual, frozen, and stored until an enriched and expanded population of CD4+/CD25+ T-reg cells may be needed. For example, the apheresis product may be cryopreserved until approximately 21 days prior to the desired time of administration of the CD4+/CD25+ T-reg cells to the individual.

To obtain a CD4+/CD25+ enriched T-reg cell population from an apheresis product, a two-step selection protocol may be used. Initially, a negative selection step may be used to remove CD8+ and CD19+ cell populations. Removal of CD8+/CD19+ cell populations is required since it eliminates the presence of these cell populations during the ex vivo expansion of the T-reg cells. The CD8+/CD19+ populations can result in the outgrowth of “effector” cells that could result in organ rejection and negate the potentially beneficial outcomes of using T-reg cells for the induction of immune tolerance. Subsequently, a positive selection step for CD4+/CD25+ cells is performed to capture only the T-reg cells. The resultant CD4+/CD25+ enriched cells may then be expanded in culture by stimulating the cells with CD3/CD28 microbeads. Expansion of the enriched CD4+/CD25+ T-reg cell population increases the T-reg cell population by about 10 fold to about 40 fold, or about 20 fold to about 80 fold, or about 40 fold to about 200 fold, or about 10 fold, or about 20 fold, or about 30 fold, or about 40 fold, or about 60 fold, or about 80 fold, or about 100 fold, or about 200 fold, or greater.

In one embodiment, a subject may be treated with a cellular therapy product derived according to the present disclosure for suppressing the subject's immune system. For example, an apheresis product may be taken from the subject, enriched and expanded for about 21 days in culture as disclosed herein, harvested, and administered fresh (without cryopreservation) back to the patient. Without being bound by theory, it is believed that application of the enriched and expanded cells may suppress the immune system of the individual to inhibit or eliminate the generation of immune processes that lead to immunological based rejection of the transplanted organ or other immune system intolerance. The cell therapy products of the disclosure balance the immune system to create tolerance to either organ transplant (eliminates organ rejection) or ameliorate autoimmune disorders with the same mechanism of action. Accordingly, the disclosure provides a method for generating an immunosuppressive effect in a mammal having an alloresponse or autoimmune response. The method comprising administering to the mammal an effective amount of the cellular therapy product described herein. In one embodiment, the mammal having an alloresponse or autoimmune response follows tissue transplantation, and wherein the method for generating an immunosuppressive effect in a mammal further comprises suppressing, blocking or inhibiting graft-vs-host disease in the mammal (e.g., human). Accordingly, the disclosure also provides a method for preventing an alloresponse or an autoimmune response in a mammal by administering to the mammal, prior to onset of an alloresponse or autoimmune response, an effective amount of the cell therapy product to prevent said response.

Administration may be through any means generally accepted for the administration of cells within an individual (e.g., intravenously).

In another embodiment, an enriched and expanded cell population may be frozen prior to administration. Accordingly, following enrichment and expansion, the cells may be frozen, thawed when needed, and then subsequently administered. It is also contemplated that the frozen expanded and enriched cell population may be frozen, and then re-enriched and reexpanded and then administered to the patient.

In one embodiment, expanded and enriched CD4+/CD25+ T-reg cell populations as disclosed herein are intended to be used as a therapeutic agent for the donor of the apheresis product from which the cells were derived. Alternatively, it is also contemplated that the therapeutic agent may be used for another individual in need thereof. It is also contemplated that such therapeutic agents may be used in multiple individuals in need thereof. It is further contemplated that further selection may be made of the T-reg cell populations to reduce the risk of rejection or other complications in an individual caused by cells donated by another.

In another embodiment, it is contemplated that the materials and methods described herein may be effectively used for enrichment and expansion of other cell subsets (e.g., not CD4+/CD25+ T-reg cells) from apheresis products or other cell sources by selecting for and against different clusters of differentiation (CDs) or other markers on the cell surface. Non-limiting examples of other potential cells include: cytotoxic T cells (CD8+) which could be used for generation of CAR-T therapies; CD34+ stem cells used for stem cell transplantation or gene-modified stem cell therapy manufacturing; dendritic cells (CD80+) and monocytes

(CD14+), which could be used for antigen-presentation directed therapies; and B cells (CD19+) for antibody-dependent cell-mediated cytotoxicity (ADCC) directed therapies, and others.

The present disclosure also provides kits containing one or more components described herein, including, for example, a thawing buffer, and/or a declumping buffer. Further, the kits may also include an enriched and/or expanded population of desired cells. Kits contemplated herein may also include a set of instructions instructing a user how to use the kit for obtaining a desired cell population from and/or administering a desired cell population to an individual. For example, one contemplated kit includes materials necessary for performing the methods described herein. In another example, a contemplated kit includes a population of enriched and expanded CD4+/CD25+ T-reg cells for administration to an individual.

In one embodiment, the disclosure is directed to a method for selecting CD25+ T-regulatory cells from an apherisis product. Fresh or frozen apherisis product may be used as a starting material. When a frozen product is used, the method includes thawing a cryopreserved apheresis product comprising T-cells. The thawed product is washed in a buffer comprising Human Serum Albumin (HSA), Magnesium Chloride (MgCl₂), and Dornase alfa. Dornase alpha is a biosynthetic form of human deoxyribunuclease I (DNase I) enzyme and is commerically available under the tradename PUILMOZYME®. The method also includes incubating the apheresis product (fresh or thawed) with one or more capture surfaces comprising a binding agent for CD8+ and CD19+ cells. Such capture surfaces are commerially available, for example, as CLINIMACS® system reagents (Milteni Biotec). After capturing the CD8+/CD19+ cells on the surfaces, a CD8/CD19 depleted product can be collected by washing the one or more surfaces with the buffer. The CD8/CD19 depleted product can then be combined with a capture surface for CD25+ cells (e.g.,)CLINIMACS° . Cell captured on the capture surface for CD25+ cells can be eluted with the buffer to provide a CD25+ enriched product. During each of the foregoing steps, one or more buffers including HSA, MgCl₂, and Dornase alfa can be used to wash or elute the surfaces and collect the cells.

In one aspect, the disclosure is directed to a composition including a thawed, previously cryopreserved apheresis product comprising T-cells and a buffer including HSA, MgCl₂, and Dornase alfa. In addition, the disclosure is directed a CD8/CD9 depleted product produced from the thawed, previously cryopreserved apheresis product. The disclosure also includes a composition comprising the thawed, previously cryopreserved, apheresis product and one or more capture surfaces for CD8+/CD19+ cells.

In another embodiment, the disclosure is directed to a method for selecting and expanding a population of CD4+/CD25+ T-regulatory cells. The method includes thawing a frozen apheresis sample received from an individual, selecting a population of CD4+/CD25+ T-regulatory (T-reg) cells from the thawed apheresis sample; and culturing the selected population of CD4+/CD25+ T-reg cells to produce an expanded population of CD4+/CD25+ T-reg cells. In accordance with the method, the expanded population of CD4+/CD25+ T-reg cells is larger than the selected population of CD4+/CD25+ T-reg cells by a factor of about 40, about 80, about 120, about 160, about 200, or about 240. The method can provide a suitable population of cells between 1×10E9 to about 5×10E9 cells for use in treating an organ transplant patient, such as a solid organ transplant (SOT) patient. When the population is expanded according to the method of the disclosure, a percentage of CD4+ T-reg cells in the expanded population of CD4+/CD25+ T-reg cells may differ from a percentage of CD4+ cells in an expanded population of CD4+/CD25+ T-reg cells selected from a fresh, non-frozen apheresis product by less than about 1 to about 10%, for example, about 1, 2, 3, 4, 5, 6, 7, 8, 9, or 10%. In addition, a percentage of CD25+ T-reg cells in the expanded population of CD4+/CD25+ T-reg cells may differ from a percentage of CD25+ cells in an expanded population of CD4+/CD25+ T-reg cells selected from a fresh, non-frozen apheresis product by less than about 1 to about 10%, for example, about 1, 2, 3, 4, 5, 6, 7, 8, 9. or10%.

In another embodiment, the disclosure is directed to a method for enriching and expanding CD4+/CD25+ T-regulatory (T-reg) cells from a cryopreserved apheresis sample. The method includes thawing the apheresis sample and suspending the thawed sample in a buffer comprising Human Serum Albumin (HAS), Magnesium Chloride (MgCl₂), and Dornase alfa. A population of CD4+/CD25+ T-regulatory (T-reg) cells from the suspended apheresis sample to selected to produce a selected population. The selected population is then cultured to produce an expanded population of CD4+/CD25+ T-reg cells. The expanded population of CD4+/CD25+ T-reg cells may be larger than the selected population of CD4+/CD25+ T-reg cells by a factor of about 10, 20, 30. 40 or 50.

In another embodiment, the method of the disclosure is directed to expanding a CD25+ cell population. The method includes culturing CD25+ cells (which may be prepared according to the previous disclosure) in a growth media supplemented with Interleukin-2 (IL-2), rapamycin, and Transforming Growth Factor Beta (TGF-β). One aspect of the disclosure includes a composition comprising the cells and the supplemented growth media. The cells may be suspended with the growth media in the presence of one or more surfaces comprising an anti-CD3+ antibody and anti-CD28+ antibody for about one to three days, for instance, about two days. Following about two days, additional IL-2 is added to the growth media, and the cells may be cultured for about another two to four days, for example, about three days. Next, additional growth media including IL-2, rapamycin, and TGF-β is added to the culture for about one to three days, for example, about two days. Then, additional growth media and IL-2, rapamycin, TGF-β, and one or more surfaces comprising an anti-CD3+ antibody and anti-CD28+ antibody are added to the culture for about one to three days, for example, about two days. Afterwards, additional IL-2, rapamycin, and TGF-β are added, and the cells are cultured for about two to four days, for example, about three days. Next, additional IL-2 is added, and the cells are cultured for about one to three days, for example, about two days. Next, additional growth media, IL-2, and TGF-β are added to the culture for about two to four days, for example, about three days. Subsequently, additional IL-2 is added to the culture for about two days. The total time for the culture according to the foregoing procedure may be about 20-22 days, for example about 21 days. The amount of IL-2, rapamycin, TGF-β added depends on the size of the culture and one of skill in the art could readily extrapolate the amount of reagents for culturing the cells from the examples following below.

Once the culture is complete, the cells are separated from the one or more capture surfaces to provide an expanded CD25+ cell population, In particular embodiments, no additional rapamycin is added to the cells beyond about 8-15 days of culture, for example, after about 8, 9, 10, 11, 12, 13, 14 or 15 days of culture.

Kits and compositions of the disclosure can include the cells, reagents, mixtures, and cultures described herein. For instance, in one embodiment, the disclosure is directed to a kit for providing an expanded and enriched CD4+/CD25+ T-reg cell population. The kit includes a buffer comprising HSA, MgCl₂, and Dornase alfa and instructions for use. A composition can include ingredients of the kits and a thawed, previously frozen, apheresis product. The composition may include a selected cell population that is in the process of being expanded or is expanded as described herein. For instance, a composition may include a population of CD4+/CD25+ cells produced from a frozen apheresis product, wherein the population is depleted of CD8/CD9 cells and is cultured in a medium including Interleukin-2 (IL-2), rapamycin, and Transforming Growth Factor Beta (TGF-β). This population is capable of producing about 1,000,000,000 to about 5,000,000,000 cells, for example, about 1,000,000,000 cells, about 2,000,000,000 cells, about 3,000,000,000 cells, about 4,000,000,000 cells, or about 5,000,000,000 cells, for infusion into a patient to ameliorate or prevent rejection of an organ transplant or ameliorate an autoimmune disease.

In another embodiment, the disclosure is directed to a method for treating a patient that has had a solid organ transplant. The method includes administering to the patient the population of cells as described herein, in particular, a population of cells that has been selected and expanded from a frozen apheresis product. Similarly, the disclosure is directed to a method of treating an autoimmune disease. The method includes administering to the patient the population of cells as described herein, in particular, a population of cells that has been selected and expanded from a frozen apheresis product.

The disclosure will be further characterized in the following examples, which do not limit the scope of the disclosure described in the claims. The Examples that follow are illustrative of specific embodiments of the disclosure, and various uses thereof. They are set forth for explanatory purposes only, and are not to be taken as limiting. The following examples establish protocols for successful enrichment, expansion, and use of therapeutically relevant populations of CD4+/CD25+ T-reg cells from frozen apheresis samples.

### EXAMPLES

### Example 1

### Selection of CD4+ and CD25+ T-Regulatory Cells

Overview: This example establishes the selection protocols for successful enrichment of CD4+/CD25+ T-reg cells.

Declumping Buffers

Three 1 L CLINIMACS® PBS/EDTA buffer bags were labeled (Bags #1, #2, and #3) and prepared by adding 20 mL of 25% human serum albumin (HSA) and 1.8 mL of 200 mg/mL MgCl₂. To Bag #1, 54 mL of 1 mg/mL PULMOZYME° (dornase alpha) was added. To each of Bags #2 and #3, 10.5 mL of 1 mg/mL PULMOZYME® was added. From Bag #1, 200 mL of solution was transferred to a sterile culture bag, which was labeled as Bag #1A, and supplemented with 45 mL 25% HSA. The approximate volumes, percentages, and/or final concentrations of each component in the Bags are shown in Table 1.

TABLE 1
Declumping Buffer Bag Components

| Bag | Bag #1A | Bag #1 | Bag #2 | Bag #3 |
|---|---|---|---|---|
| (total volume) | (245 mL) | (876 mL) | (1032 mL) | (1032 mL) |
| HSA | 5% | 0.5% | 0.5% | 0.5% |
| MgCl₂ | 2.9 mM | 3.5 mM | 3.5 mM | 3.5 mM |
| PULMOZYME ® | 41 U/mL | 50 U/mL | 50 U/mL | 50 U/mL |

Leukapheresis Product Cryopreservation

A collected leukapheresis sample was centrifuged at 2800 rpm for 11 minutes. Using a plasma extractor and an electronic scale, the supernatant was removed (i.e., plasma was expressed). Plasma was added back to the centrifuged sample to reach a volume of 60 mL. The leukapheresis sample was then transferred to a cryopreservation bag. A freezing solution of 50% DMSO (dimethyl sulfoxide) in Normal Saline (0.9% sodium chloride) was prepared, then placed in a 2-8° C. refrigerator for at least 10 minutes. The 50% DMSO freezing solution was added to the cryopreservation bag to reach a final DMSO concentration of 10%.

The cryopreservation bag was frozen in a controlled-rate freezer at an average cooling rate of 1° C./min, to an endpoint of −100° C. Once frozen, the bag was transferred to a monitored liquid nitrogen freezer and stored in the vapor phase.

Leukapheresis Product Thawing

Thawing of apheresis products generally results in some cellular clumping with associated decreased viability. Column separation of thawed apheresis products can be problematic due to the clumping of the product and the potential loss of cellular starting material. Therefore, a “thawing buffer” consisting of a PBS/EDTA buffer (phosphate—buffered saline, pH 7.2, supplemented with 1 mM EDTA), such as CliniMACS® PBS/EDTA buffer (Miltenyi Biotec), 5% human serum albumin, 3.5 mM MgCl₂ and 50 U Pulmozyme®/mL was used for the initial washing of the thawed apheresis product as well as for the selection process.

The cassette holding the frozen leukapheresis bag was removed from the dry shipper and immersed completely in a 37±2° C. water bath. Once the cells thawed, but were still cold, the leukapheresis bag was removed from the cassette.

CD8/CD19 Negative Selection

Thawed leukapheresis cells were transferred to a 600 mL TRANSFER PACK™ container (FENWAL™, Lake Zurich, Ill.). The entire contents of Bag #1A were added to the container. The container was incubated for 30 minutes, and then centrifuged at 1800 rpm for 15 minutes, all at room temperature. Using a plasma extractor and an electronic scale, the supernatant was removed and discarded. An aliquot of 200 mL of buffer from Bag #1B was added to the container. The container was centrifuged at 1800 rpm for 15 minutes at room temperature. Using a plasma extractor and an electronic scale, the supernatant was removed and discarded. The final product volume was adjusted to 85±5 mL using buffer solution from Bag #1B. The cells were resuspended by gently agitating the container.

The entire contents of a CLINIMACS® CD8 microbead kit and the CD19 microbead kit were added to the resuspended cells. The microbead kits include a colloid of magnetic antibodies specific to the cells of interest (e.g., CD8+ CD19+ cells) and Iron-Dextran. The container was mixed gently before incubating for 30 minutes at room temperature on a rotator at 25 rpm. The incubated cells were diluted to 450 mL with buffer solution from Bag #1B. The container was centrifuged at 1800 rpm for 15 minutes. Using a plasma extractor and an electronic scale, the supernatant containing excess microbeads was removed and discarded. The product volume was adjusted to 100±2 mL with buffer solution from Bag #1B. The cells were resuspended by gently agitating the container.

The container and Buffer Bag #2 were connected to a CLINIMACS® Plus instrument, and a CD8/CD19 depletion program was executed, which separates CD8+/CD19+ cells using a high-gradient magnetic separation column (Miltenyi). The magnetically-labeled CD8+CD19+ cells are retained in the magnetized column and separated from the unlabeled cells. The unlabeled cells are eluted out of the column and consist of a cell population depleted of CD8+/CD19+ cells. The CD8/CD19 depleted cells were collected in a “CD8/19 Depleted Product Preparation” Bag.

CD4/CD25 Enrichment

The CD8/CD19 depleted cells were diluted with cold (4-8° C.) buffer (Buffer Bag #3) to a total volume of 380 mL. The cells were resuspended by gently agitating the CD8/19 Depleted Product Preparation Bag. The diluted product was cooled to 4-8° C. in a refrigerator for 20 minutes.

The entire contents of a CLINIMACS® CD25 microbead kit were combined with the diluted, cooled CD8/CD19 depleted cells in the CD8/19 Depleted Product Preparation Bag and the combination was mixed gently before incubating for 15 minutes at 4-8° C. on a rotator at 25 rpm. The incubated cells were transferred to a 600 mL TRANSFER PACK™ container, and then diluted with buffer solution from Bag #3. The container was centrifuged at 1800 rpm for 15 minutes at room temperature. Using a plasma extractor and an electronic scale, the supernatant containing excess Microbead reagent was removed and discarded. The product volume was adjusted to 100±2 mL with buffer solution from Bag #3. The cells were resuspended by gently agitating the container.

The container and Buffer Bag #3 were connected to a CLINIMACS® Plus instrument, and a CD4/CD25 enrichment program was executed. This protocol is similar to that described above, with the exception that the desired CD4+/CD25+ cells are selected for and undesired cells are washed out. CLINIMACS® Magnetic Cell Separation Systems separate mixed cell populations in a magnetic field using an immunomagnetic label specific for the cells of interest (e.g., CD4+/CD25+ (bright), referred to as regulatory T-cells, or T-Reg). Thus, once the unlabeled cells were removed from the column, the retained cells (CD4+/CD25+ cells) were eluted by removing the magnetic field from the column, washing the cells out and collecting them. The resulting CD4+/CD25+ enriched cells were collected in a “CD25 Enriched Cell” Bag. Results

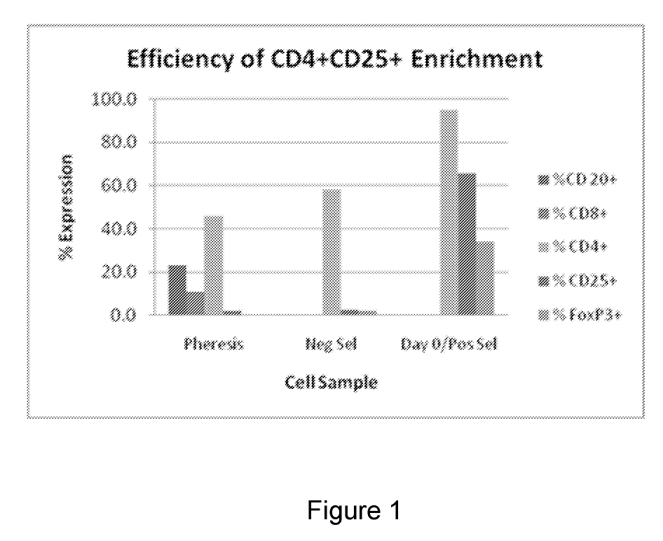

Flow cytometry was used to determine the phenotypic characteristics of the selected population. The selection protocol yielded 98.7% CD4+ cells, 86.8% CD25+ cells, 0.0% CD8+ cells, and 0.1% CD20+ cells (see, Table 2 “Day 0,” which is a representative data set and FIG. 1, which includes the data shown in Table No. 2). FoxP3+ was used as a separate marker for T-reg cells.

TABLE 2
CD4+/CD25+ Enriched Cells

| | Day 0 |
|---|---|
| CD4+ | 98.7% |
| CD25+ | 86.8% |
| FoxP3+ | 44.0% |
| CD8+ | 0.0% |
| CD20+ | 0.1% |
| Viability | 94.9% |
| Cell # | 1 × 10⁷ |
| Sterility | negative |

The data in Table No. 2 demonstrate that the combined selection processes used for selecting T-reg cells were successful.

### Example 2

### T-Regulatory Cell Expansion

Overview: Enriched populations of T-reg cells obtained in Example No. 1 are expanded in the present example to provide therapeutically relevant numbers of T-reg cells.

Preparation of Cell Media

Growth medium was prepared by adding 100 mL 5% heat-inactivated AB serum (Valley Biomedical, Winchester, Va.) to 1900 TexMACS medium (Mitlenyi Biotec, San Diego, Calif.). A sample of 2.2×10⁷ IU of IL-2 (Prometheus Laboratories, San Diego, Calif.) was reconstituted in 1 mL of sterile water, then diluted with 9 mL growth medium to a final concentration of 2.2×10⁶ IU/mL (“diluted IL-2”). An aliquot of 1.5 mL of 2.5 mg/mL Rapamycin (Sigma-Aldrich, St. Louis, Mo.) was diluted with 1.35 mL growth medium to a final concentration of 0.25 mg/mL (“diluted rapamycin”). To 1 mL 5% acetic acid, 20 mL sterile water was added to reach a final concentration of 40 mM. A 100 μg sample of TGF-β (Invitrogen, Carlsbad, Calif.) was reconstituted in 1 mL 40 mM acetic acid, then diluted with 8.77 mL TexMACS buffer and 0.23 mL AB serum, to a final concentration of 10 μg/mL. Reconstituted and diluted TGF-β was stored in 0.5 mL aliquots frozen at −20° C. As needed, TGF-β aliquots were thawed and diluted with 4.5 mL growth medium to a final concentration of 1 μg/mL, and kept refrigerated at 2-8° C. Complete Growth Meeting (GM) was prepared by adding 900 μL diluted IL-2 solution, 800 μL diluted Rapamycin solution, and 2.0 mL thawed, diluted TGF-β solution to 2 L growth medium.

Cell Expansion Protocol

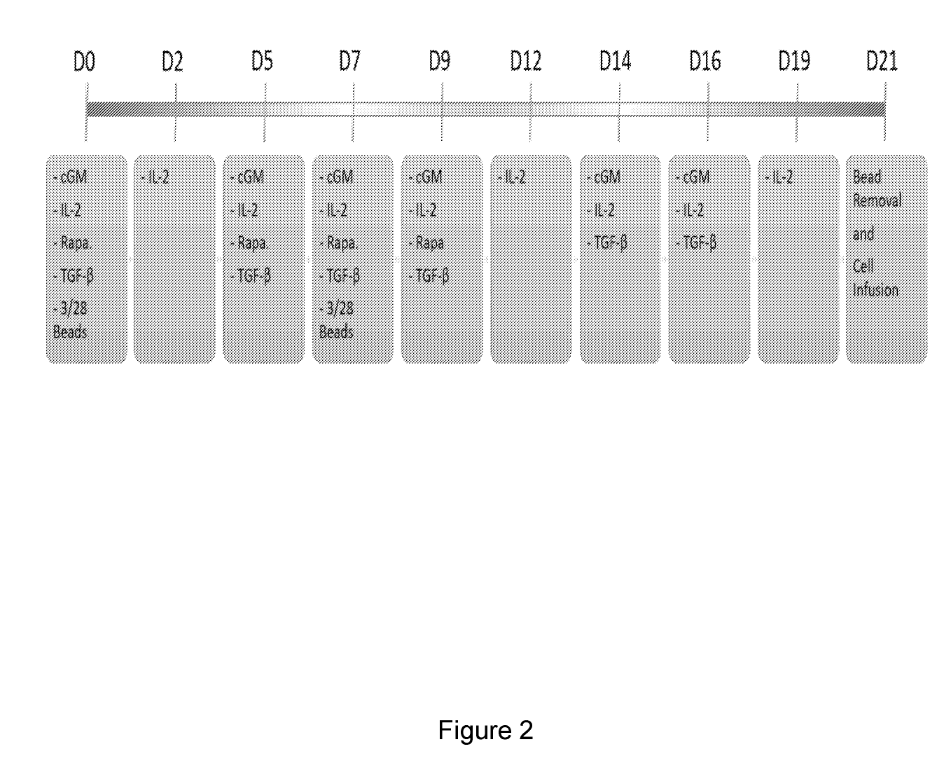

A population of CD4+/CD25+ enriched cells from Example No. 2 was split into fractions containing 3×10⁷ total nucleated cells each in separate culture flasks (G-Rex100M, Wilson Wolf Manufacturing, New Brighton, Minn.) and diluted with 450 mL GM. CD3/CD28 ExpAct beads (Miltenyi) (0.6 mL, 2×10⁸ beads/mL) was added to each flask to reach a 4:1 bead:cell ratio. GMP ExpAct® beads are composed of MACSi® Beads that have been coated with CD3 and CD28 antibodies. These beads provide non-specific stimulation signals required for the expansion of the T-reg cell population. The culture flasks were then incubated at 37° C., 5% CO₂ throughout a 21-day expansion protocol as described below and illustrated in FIG. 2:

On Day 2 (D2), 200 μL diluted IL-2 solution was added to each flask.

On Day 5, 50 mL GM, 225 μL diluted IL-2 solution, 200 μL diluted Rapamycin, and 500 μL thawed, diluted TGF-β solution were added to each flask.

On Day 7, a sample was taken from each culture flask and a manual cell count was performed. Following sample collection, 50 mL GM, 250 μL diluted IL-2 solution, 220 μL diluted Rapamycin, and 550 μL thawed, diluted TGF-β solution were added to each flask. CD3/CD28 ExpAct bead solution was added to the culture flasks at a 1:1 bead:cell ratio, based on the manual cell count.

On Day 9, 50 mL GM, 272 μL diluted IL-2 solution, 240 μL diluted Rapamycin, and 600 μL thawed, diluted TGF-β solution were added to each flask. No further Rapamycin was added to the cultures after Day 9.

On Day 12, 272 μL diluted IL-2 solution was added to each flask.

On Day 14, aliquots were collected of the supernatant from each flask for sterility testing. Cells in each flask were then resuspended, and aliquots were collected for in-process testing. After aliquot collection, 100 mL GM, 318 μL diluted IL-2 solution, and 700 μL thawed, diluted TGF-β solution were added to each flask.

On Day 16, 100 mL GM, 364 μL diluted IL-2 solution, and 800 μL diluted TGF-β solution were added to each flask.

On Day 19, 364 μL diluted IL-2 solution was added to each flask.

On Day 21, cells were resuspended, and aliquots were collected for a manual cell count. After aliquot collection, CD3/CD28 ExpAct beads were removed according to a manufacturer supplied Miltenyi Protocol.

Results

The cell expansion protocol yielded a 43-fold increase in cells, highly selected for CD4+ and CD25+ cells (see, Table 3, “Day 21.” “Day 0” cells are the same as from Table No. 2 above).

TABLE 3
CD4+/CD25+ Expanded Cells

| | Day 0 | Day 21 |
|---|---|---|
| CD4+ | 98.7% | 99.5% |
| CD25+ | 86.8% | 97.6% |
| FoxP3+ | 44.0% | 46.5% |
| CD8+ | 0.0% | 1.4% |
| CD20+ | 0.1% | 0.1% |
| Viability | 94.9% | 91.8% |
| Cell # | 1 × 10⁷ | 4.3 × 10⁸ |
| Fold Expansion | — | 43 fold |
| Sterility | negative | negative |

The observed yield of enriched and expanded CD4+/CD25+ T-reg cells is considerably greater than reported in the literature. Such an increase over the yields reported in the literature was unexpected. (Indeed, in subsequent clinical manufacturing of nine cellular products following the same protocols, the average yield was 91-fold (range 29-fold to 180 fold), which further demonstrates the repeatability of such unexpected yields (data not shown)). The results further show that there was not an outgrowth of undesirable CD8+ cells and that a high purity of CD4+/CD25+ T-reg cells was retained.

### Example 3

### Expansion of Fresh vs. Cryopreserved Leukapheresis Products

Overview: this example sought to compare the efficacy of the present methodologies for CD4+/CD25+ cell selection and expansion in fresh versus frozen leukapheresis samples.

A leukapheresis product from a healthy donor was split into two samples. The first sample [frozen sample] was treated according to the procedure in Example Nos. 1 and 2, i.e., declumping buffer was prepared, and the cells were cryopreserved, thawed, T-reg cells were selected, and T-reg cells were expanded. For comparative purposes, the second sample [fresh sample] was not cryopreserved or exposed to buffer containing Pulmozyme®, but was otherwise treated according to the procedure in Example Nos. 1 and 2, i.e., the T-reg cells were selected and expanded.

Fresh vs. Frozen CD4+/CD25+ T-reg cell Immunosuppressive Capacity

The culture expanded CD4+/CD25+ T-reg cells from fresh and frozen samples were evaluated for their functional activity to suppress T cell responsiveness in a standard mixed lymphocyte proliferation assay (MLR) (see Bresatz S, Sadlon T, Millard D, Zola H, Barry S C. Isolation, propagation and characterization of cord blood derived CD4+ CD25+ regulatory T cells. J Immunol Methods 2007; 327: 53-62).

Results

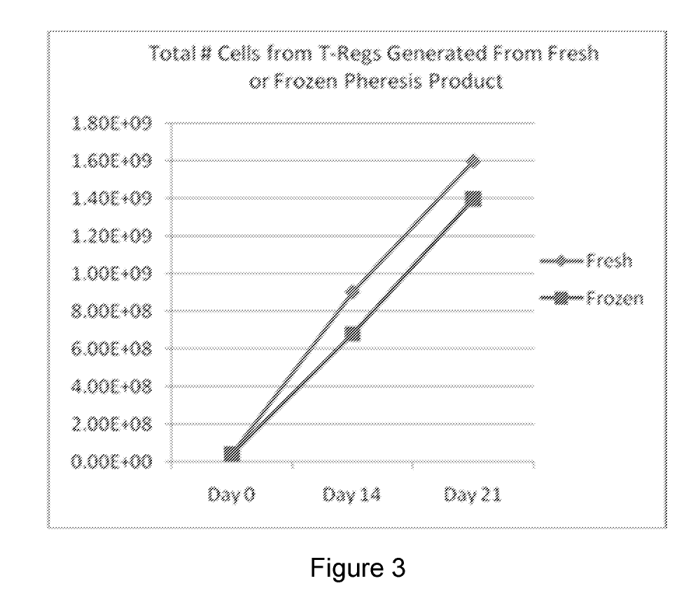

Cryopreservation and thawing with the declumping buffer before selection and expansion had a negligible effect on the final cellular product when compared to the selected and expanded fresh sample (see Table No. 4 and FIG. 3). Table No. 4 indicates the detailed characterization of the initial cell product at culture initiation and the final product of fresh versus frozen expanded cell populations. As seen in Table No. 4, characteristics of frozen cells and fresh cells are comparable both upon initiation of T-reg cell expansion and after the T-reg cells have undergone expansion.

TABLE NO. 4
Fresh vs. Frozen T-Reg Cell Products

| | Fresh | | Frozen | |
|---|---|---|---|---|
| | Day 0 | Day 21 | Day 0 | Day 21 |
| Cells loaded | 9.3 × 10⁹ | — | 9.9 × 10⁹ | — |
| Cells recovered | 4.1 × 10⁷ | — | 3.8 × 10⁷ | — |
| CD4+ | 97.8% | 99.6% | 97.4% | 97.6% |
| CD25+ | 45.0% | 99.0% | 30.7% | 94.1% |
| FoxP3+ | 6.0% | 41.4% | 2.0% | 41.7% |
| CD8+ | 0.1% | 1.3% | 0.1% | 0.0% |
| CD20+ | 0.1% | 0.1% | 0.0% | 0.1% |
| Viability | 95.4% | 96.7% | 92.6% | 97.9% |
| Cell # | 4.1 × 10⁷ | 1.6 × 10⁹ | 3.8 × 10⁷ | 1.4 × 10⁹ |
| Fold Expansion | — | 39.0 fold | — | 36.8 fold |
| Sterility | negative | negative | Negative | negative |

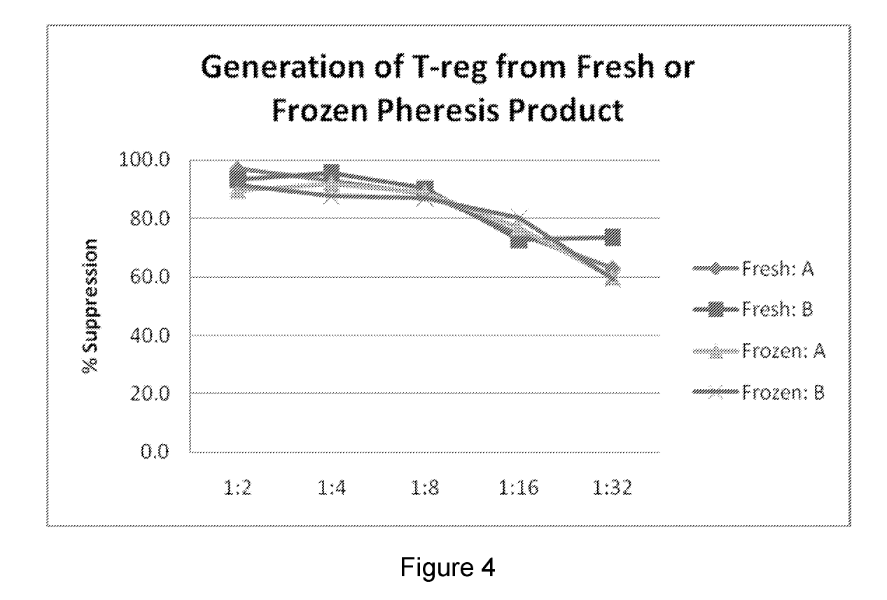

Moreover, as shown in FIG. 4, enriched and expanded CD4+/CD25+ T-reg cells from fresh and frozen sources had nearly identical immunosuppressant effects.

Therefore, these unexpected results indicate for the first time that cryopreserved leukapheresis products that are thawed using the declumping buffer described above may be effectively enriched and expanded for CD4+/CD25+ T-reg cells and are equivalent to fresh cells. Indeed, using these techniques it has been demonstrated that T-reg cells generated from fresh apheresis product are not significantly different (in terms of growth potential, immunosuppressive function, viability and phenotypic characterization) than those generated from cryopreserved apheresis product.

### Example 4

### Immunosuppressive Capacity of Enriched and Expanded T-Reg Cells obtained from Renal Failure Patients

Overview: to determine whether the enriched and expanded CD4+/CD25+ T-reg cells obtainable by the present disclosure from renal failure patients would be effective for immunosuppressive therapy. In this example, autologous apheresis products were taken from patients with renal failure who would be undergoing kidney transplants, CD4+/CD25+ T-reg cells were enriched and expanded as described above, and the enriched and expanded CD4+/CD25+ T-reg cells were tested for their immunosuppressive capacity.

T-Reg Cell Enrichment and Expansion

Apheresis products from Renal Failure Patients (defined as end-stage kidney disease patients who are undergoing living donor kidney transplantation) that were used in this T-reg cell feasibility study were obtained from consented donors under an IRB (Northwestern University) approved protocol (STU20666). The enrichment and expansion of CD4+/CD25+ T-reg cells were carried out as described above.

CD4+/CD25+ T-Reg Cell Immunosuppressive Capacity

The culture expanded CD4+/CD25+ T-reg cells from renal failure patients were evaluated for their functional activity to suppress T cell responsiveness in an MLR, as described above.

Results

Expansion, viability, and phenotypic characterization were all comparable to that observed from previous results (see Table 5).

TABLE 5
Renal Failure Patient CD4+/CD25+ T-reg Cells

| | Patient #06 | Patient #06 | Patient #01 | Patient #01 |
|---|---|---|---|---|
| | Day 0 | Day 21 | Day 0 | Day 21 |
| CD4+ | 98.7% | 99.5% | 96.3% | 99.9% |
| CD25+ | 86.8% | 97.6% | 87.4% | 99.8% |
| FoxP3+ | 44.0% | 46.5% | 59.2% | 42.2% |
| CD8+ | 0.0% | 1.4% | 0.0% | 1.9% |
| CD20+ | 0.1% | 0.1% | 0.1% | 0.1% |
| Viability | 94.9% | 91.8% | 93.3% | 93.7% |
| Cell # | 1 × 10⁷ | 4.3 × 10⁸ | 1.6 × 10⁸ | 1.1 × 10¹⁰ |
| Fold | — | 43 fold | — | 69 fold |
| Expansion | | | | |
| Sterility | negative | negative | negative | negative |

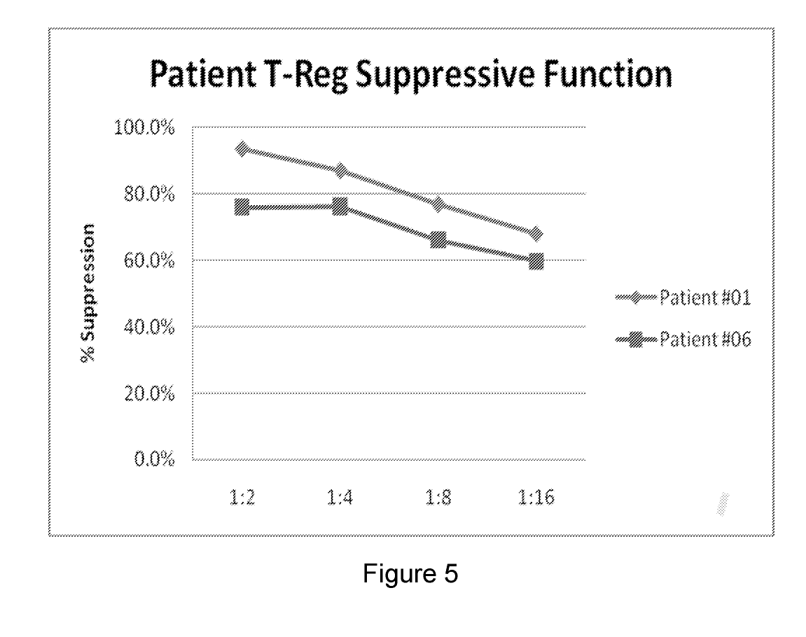

The culture expanded CD4+/CD25+ T-reg cells from renal failure patients (see FIG. 5) had suppressive activity comparable to that of T-reg cells generated from normal donors (not shown). Therefore, these data indicate that enriched and expanded CD4+/CD25+ T-reg cells from renal failure patients (i.e., individuals anticipating receiving a tissue transplant) may be useful as immunosuppressants and may be useful for treatment of rejection of allografts by host immune systems and graft versus host disease.

### Example 5

### Ex Vivo Expanded Recipient Regulatory T Cells in Living Donor Kidney Transplants

Overview:

A phase I dose-escalation clinical trial was initiated based on the above-described TReg Adoptive Cell Therapy (TRACT) for solid organ transplants (SOTs).

Subjects:

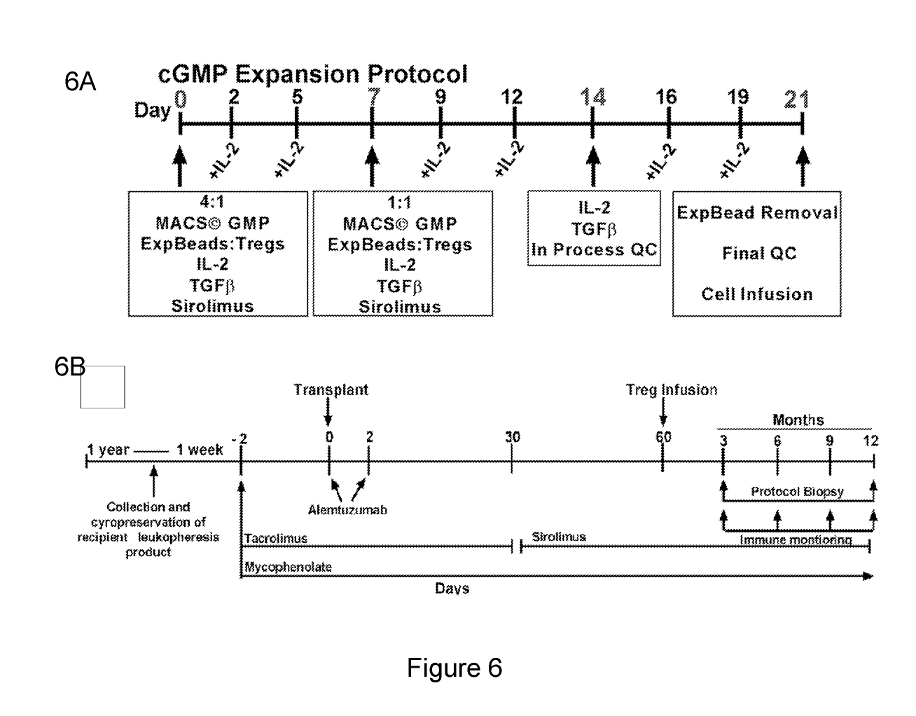

A Phase I trial of autologous, polyclonally expanded T-reg Adoptive Cell Therapy (TRACT) was initiated in living donor kidney transplant recipients. This was a nonrandomized dose-ranging study with 3 tiers of cell dosing (0.5, 1, and 5×10E9 cells infused, n=3 subjects/tier). The clinical protocol for the trial is graphically shown in FIG. 6. The inclusion and exclusion criteria were as follows:

Inclusion Criteria
18-65 years
No prior organ transplant
Single organ (kidney) recipients
Females: negative serum pregnancy test
Understand and give informed consent

Exclusion Criteria
Sensitivity; Contraindication; or non-compliance to Sirolimus, Tacrolimus or MMF
Active Infection; Severely limiting secondary diseases; psychiatric illness;
Cardiovascular disease; or addiction
Positive flow cytometric crossmatch
PRA>20%
Current or historic donor-specific antibodies
18>BMI<35
Malignancy within 3 years of transplant (non-melanoma skin cancer excluded)
HIV or HBsAg positive
WBC<4,000/mm3; platelet <1000,000/mm3; Triglyceride <400 mg/dl; total cholesterol >300 mg/dl
Anti-T cell therapy or other investigational drug within 30 days of transplant

Enrolled subjects under-went a non-mobilized leukopheresis at least two weeks prior to kidney transplant. This leukopheresis product was cryopreserved for later isolation and manufacturing of T-regs. Kidney transplant recipients received alemtuzumab induction to achieve lymphodepletion and were started on tacrolimus and mycophenolate-based immunosuppression. Subjects were converted from tacrolimus to sirolimus at 30 days post-transplant to provide an immunosuppressive milieu conducive to the survival of infused T-regs.

Ex Vivo GMP Expansion of Recipient T-regs:

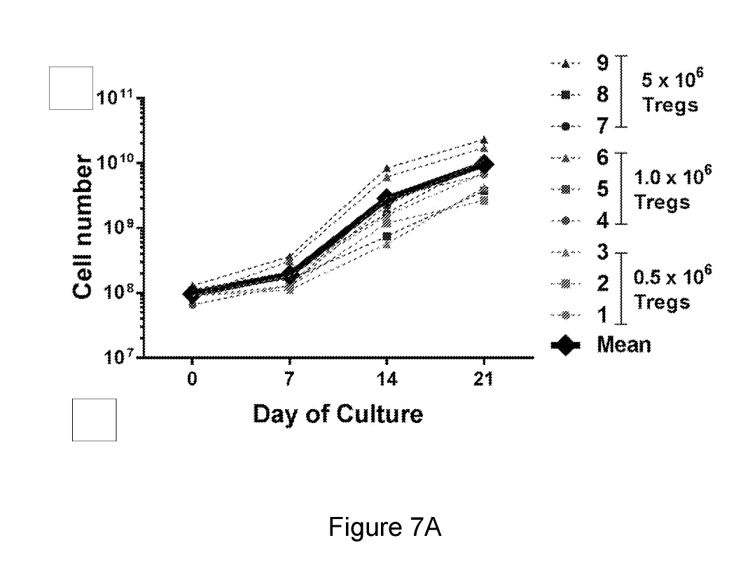

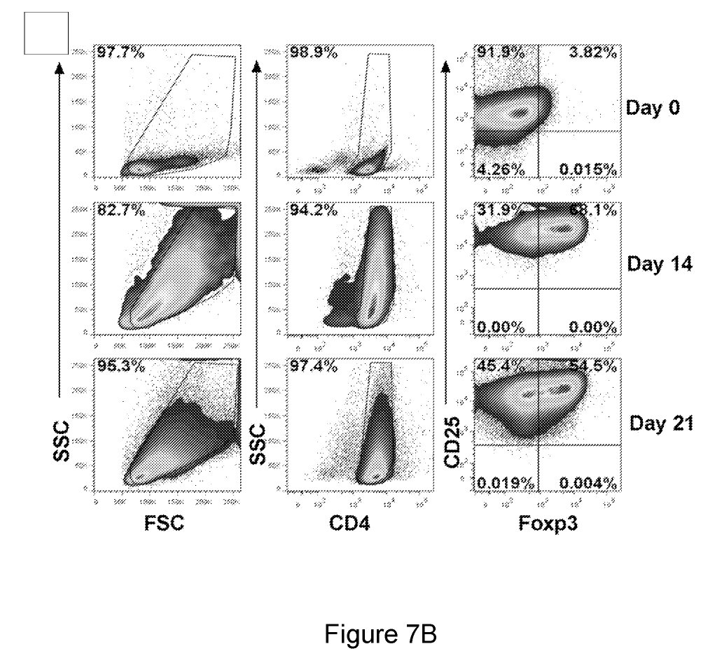

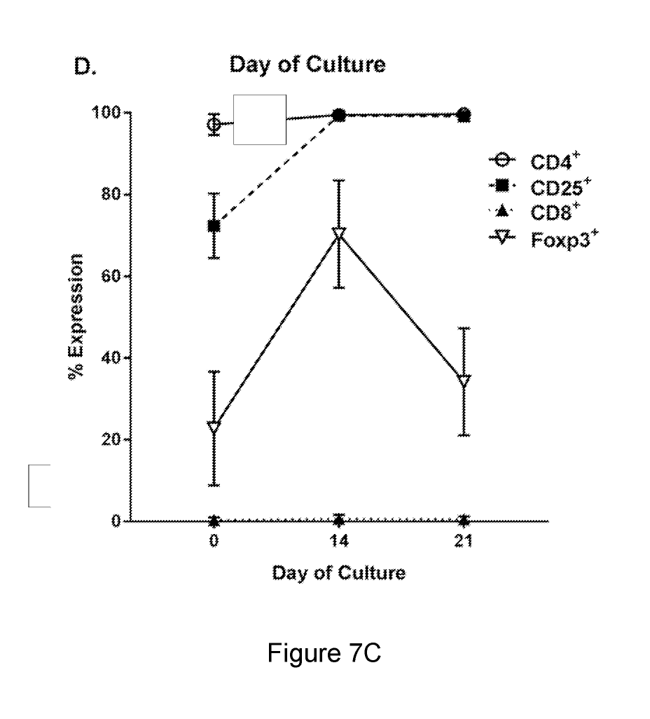

In a GMP compliant facility, CD4⁺CD25⁺ T-regs were isolated from the patient's leukopheresis products using all CliniMACS® reagents and systems, as described herein. CD127 was not included in T-reg isolation procedures due to a lack of a GMP compliant reagent. T-reg expansion began with stimulation using MACS GMP ExpBeads® and IL-2, TGFβ, and Sirolimus on days 0 and 7, Sirolimus was not added to the culture after day 9 and expansion beads were removed before infusion into recipients. See FIGS. 6A and 6B. Robust expansion was observed, and cell threshold demands were met for the dose dependent study in all nine T-reg samples, despite varying causes of end stage renal disease. FIG. 7A shows growth curves of T-reg cells (absolute number) in nine expansion cultures. Phenotypically, the expansion protocol generated a classic CD4⁺CD25⁺Foxp3⁺ with little contaminating CD8⁺ cell throughout culture (FIGS. 7B and 7C). Greater than 99% of cells were CD4+ most of which acquired CD25^{High} phenotype by day 14. However, a decrease in Foxp3 expression from day 14 to day 21 of culture was observed possibly due to the strain of rapid cellualr expansion (FIG. 7C). Despite this reduction in the Foxp3 expression, DNA methylation analyses indicated that the Foxp3 promoter was still demethylated (data not shown), suggesting the expanded cell product retained the regulatory nature. Also, this loss of Foxp3 expression did not result in any adverse clinical events.

Expansion Alters T-Reg Surface Receptor Expression:

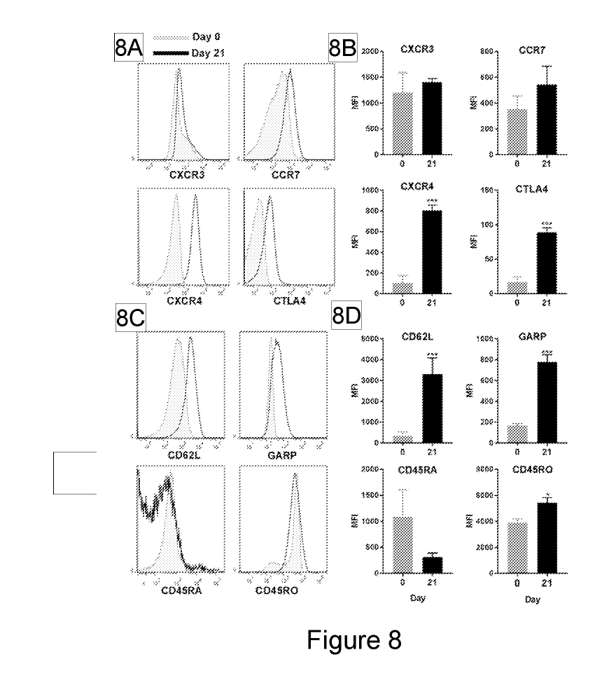

In order for T-regs to be effective and survive in vivo they must home to sites of inflammation and secondary lymphoid tissues. Therefore, the expression of key chemokine and other surface receptors from beginning to the end of T-reg expansion was characterized. An increase in CXCR3, CXCR4, and CCR7 expression from day 0 to day 21 of T-reg expansion (FIG. 8, Panel 8A and Panel 8B) was observed, suggesting the expanded T-reg product has the capability to home to sites of inflammation (CXCR3) and from the blood to the lymph nodes (CCR7). Also, an increase in CD62L was observed as the T-reg expansion progressed, displaying the ability of these T-regs to home to lymph node tissue (FIG. 8, Panels 8C and Panel 8D). There was also an increase in CTLA-4 and GARP expression by the end of culture, which are two key molecules for T-reg suppressive function. As T-reg culture progressed, there was a transition from CD45RA to CD45RO expression, suggesting the infused T-reg product displayed a more memory then naïve phenotype (FIG. 8, Panel 8C and Panel 8D).

Expanded T-Regs Retained Clonal Diversity:

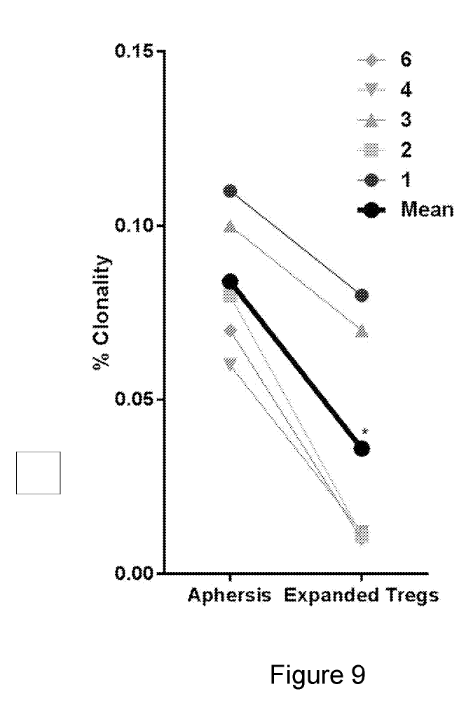

In conjunction with receptor expression, the effect of the expansion protocol on the clonal diversity of the T-reg final product was analyzed. Using high-throughput sequencing, unique TCR rearrangements were analyzed from six patient apheresis and matched final T-reg products. The clonal diversity was found to increase from initial apheresis product to the final T-reg expanded product (FIG. 9 and Table 6). This is likely due to uncovering of low frequency clones within the T-reg product that were below the detection criteria in the apheresis product, not by the generation of new clones as expansion is an ex vivo procedure. Overall, the expansion protocol generated a T-reg product that displayed key homing receptors and maintained a diverse T cell repertoire.

TABLE 6
Number of Characteristic Clones

| | Total | Apheresis | T-reg | | Top 10% |
|---|---|---|---|---|---|
| Patient | Productive | Unique | Unique | | Clone |
| Sample | Rearrange | Rearrange | Rearrange | Shared | Frequency |
| 1 | Aph: 41,618 | 38,807 | 48,216 | 2,811 | Aph: 8.03 |
| | T-reg: 51,027 | | | | T-reg: 2.25 |
| 2 | Aph: 77,589 | 71,434 | 49,297 | 155 | Aph: 3.87 |
| | T-reg: 49,452 | | | | T-reg: 0.31 |
| 3 | Aph: 8,246 | 7,983 | 44,426 | 263 | Aph: 15.64 |
| | T-reg: 44,689 | | | | T-reg: 2.11 |
| 4 | Aph: 60,904 | 60,576 | 62,639 | 328 | Aph: 5.54 |
| | T-reg: 62,967 | | | | T-reg: 0.45 |
| 6 | Aph: 60,098 | 59,789 | 103,373 | 309 | Aph: 5.96 |
| | T-reg: 103,682 | | | | T-reg: 0.16 |

Expanded T-Regs were Potently Suppressive and Induce Infectious Tolerance:

The suppressive function of the T-reg products was analyzed on days 0, 14 and 21 by using a classical mixed lymphocyte reaction (MLR) and measuring thymidine incorporation. T-regs were used as modulators in mixed lymphocyte reaction of autologous PBMC (R) stimulated with allogeneic irradiated PBMCs (Sx). Additional responder PBMC (Rx) was used as control modulators. After 7 days, a standard thymidine incorporation assay was performed.

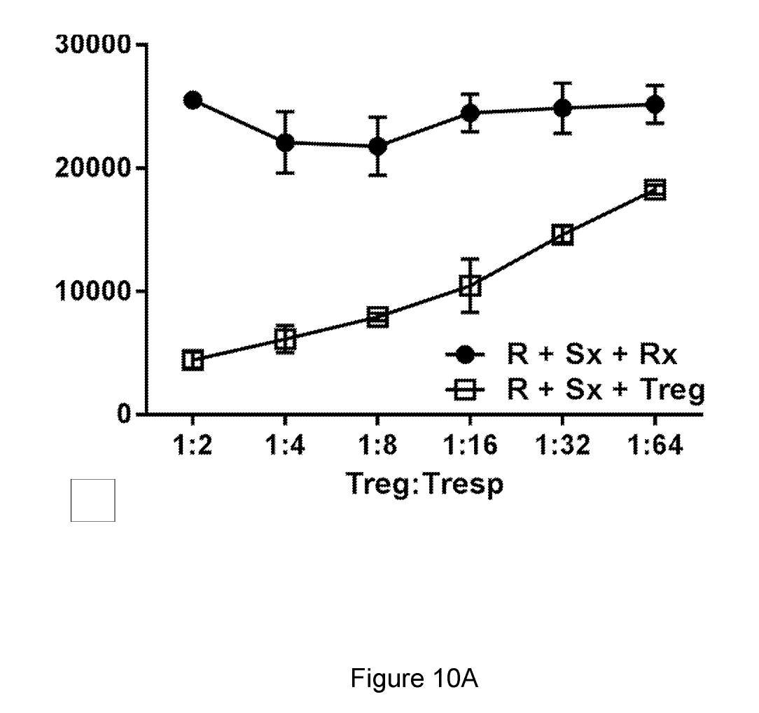

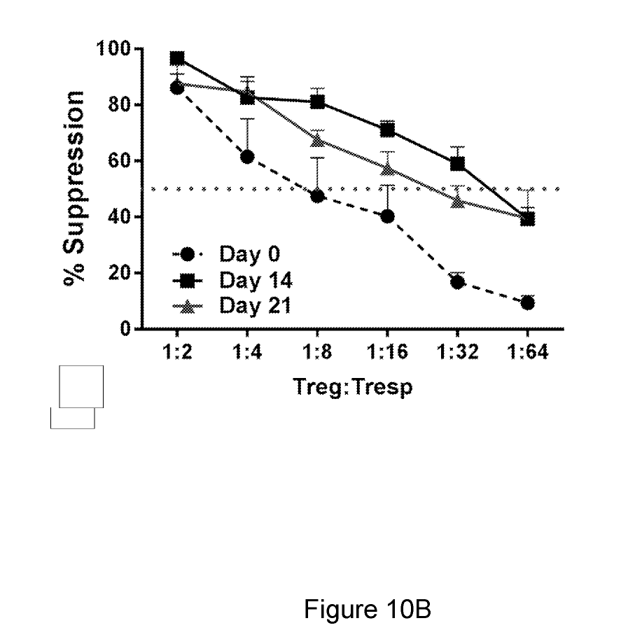

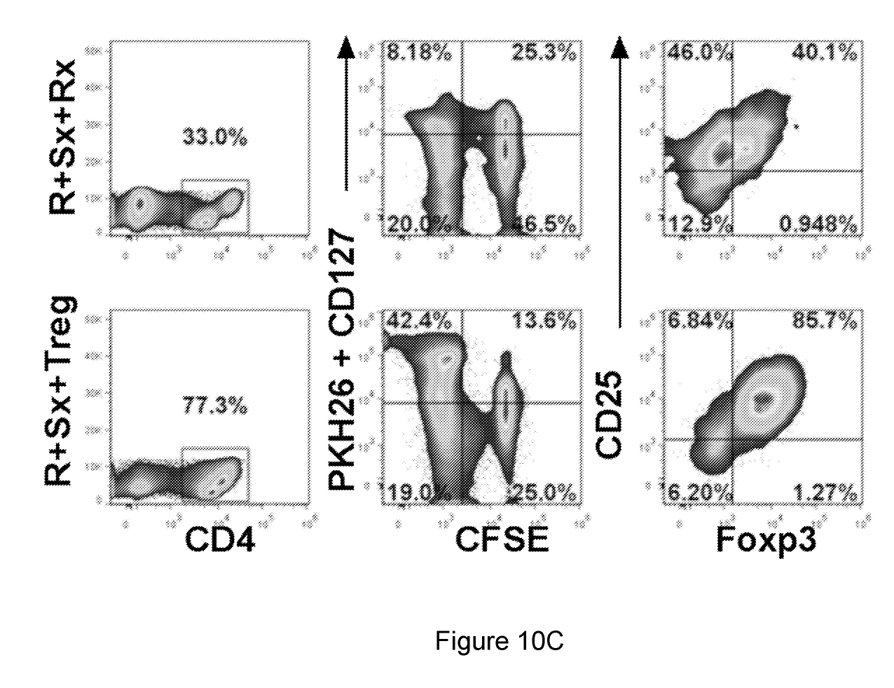

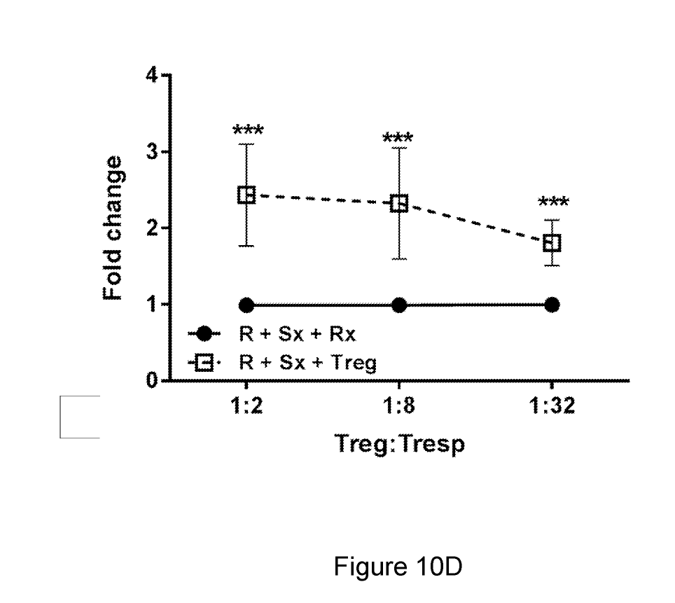

An increase in suppressive function in a dose dependent manner was observed as the T-regs expanded as shown by eight fold fewer Day 21 T-regs needed to achieve 50% suppression compared to Day 0 T-regs (See FIGS. 10A and 10B.) FIG. 10B shows a mean ±SD percentage of suppression that were calculated for each individual experiment (n=9) using the formula described in Levitsky J, et al., Transplantation, 2009, 88(11):1303-11.

One mechanism of T-reg mediated suppression is through the generation of new T-regs from naive T cells, a process known as infectious tolerance. Therefore, an in vitro assay was developed to test the potential of the T-reg product to induce infectious tolerance. Briefly, MLRs were performed with recipient responder PBMCs that were labeled with CFSE and donor irradiated stimulator PBMCs labeled with PKH-26 in presence of T-regs that were also labeled with PKH-26. Seven days later the percentage of CD4⁺CD127⁻CD25⁺Foxp3⁺ cells derived from the CFSE⁺PKH-26⁻ recipient PBMCs that proliferated (diluted CF SE) was calculated (FIG. 10C). A significant increase in the percentage of CD4⁺CD127⁻CD25⁺Foxp3⁺ induced from the recipient PBMCs was observed when the T-reg product was present compared to baseline (no T-regs present) (FIG. 10D).

Expanded T-Regs Met Release Criteria:

The expanded T-regs underwent additional extensive testing to determine microbial sterility, endotoxins, cell viability, phenotypic characteristics, and number of residual beads used for stimulating the cells. It was found that the resultant cells met all the release criteria; i.e., negative aerobic, anaerobic and fungal contaminations, negative mycoplasma and negative gram stain; <5.0 EU/kg endotoxin; >70% viable; >70% CD4⁺ CD25⁺; <10% CD8⁺ and CD19⁺; <3000 Exp-Act® beads/10E8 cells (Table 7).

TABLE 7

| | | | Time | |
|---|---|---|---|---|
| | | | Testing | Results |
| | | | Is | available |
| | | | Preformed | prior to |
| Test | Method | Release Criteria | (Day) | release |
| Bacteriologic | Bac Tee BD aerobic | Negative | 14 and 21 | No |
| sterility | and anaerobic system | | | |
| Gram Stain | Micro Lab SOP | Negative | 14 and 21 | Yes |
| Mycoplasma | E-MYCO ™ PCR | Negative | 14 and 21 | No |
| detection | | | | |
| Endotoxin | EndoSafe | <5.0 EU/kg of body | 14 and 21 | Yes |
| detection | | weight per dose | | |
| Viability | 7AAD staining | >70% viable | 14 and 21 | Yes |
| | Flow | | | |
| Expression of | CD4⁺CD25⁺ | >70% CD4⁺CD25⁺ | 14 and 21 | Yes |
| T-reg Markers | CD3⁺CD8⁺CD19⁺ | <10% CD3⁺CD8⁺CD19⁺ | | |
| | Flow | | | |
| Residual Exp- | Miltenyl Bead Count | <3000 beads/1 × 10⁸ cells | 21 | Yes |
| Act Bead | Assay | | | |
| Concentration | | | | |
| Potency | MLR suppression | >50% suppression | 14 and 21 | No |
| (In process | Assay | @ 1:2 (T-reg:Tresp) | | |
| testing on Day | | | | |
| 14) | | | | |

In addition, potency was tested using MLR inhibition assays on days 14 and 21 of the culture and observed the expanded T-regs exceeded the expected regulatory capabilities. The day 14 assay was the in process testing and the data were available before the infusion of the expanded T-regs into the recipients.

Infusion of Expanded T-Regs into Kidney Recipients resulted in Amplification of T-Regs In Vivo:

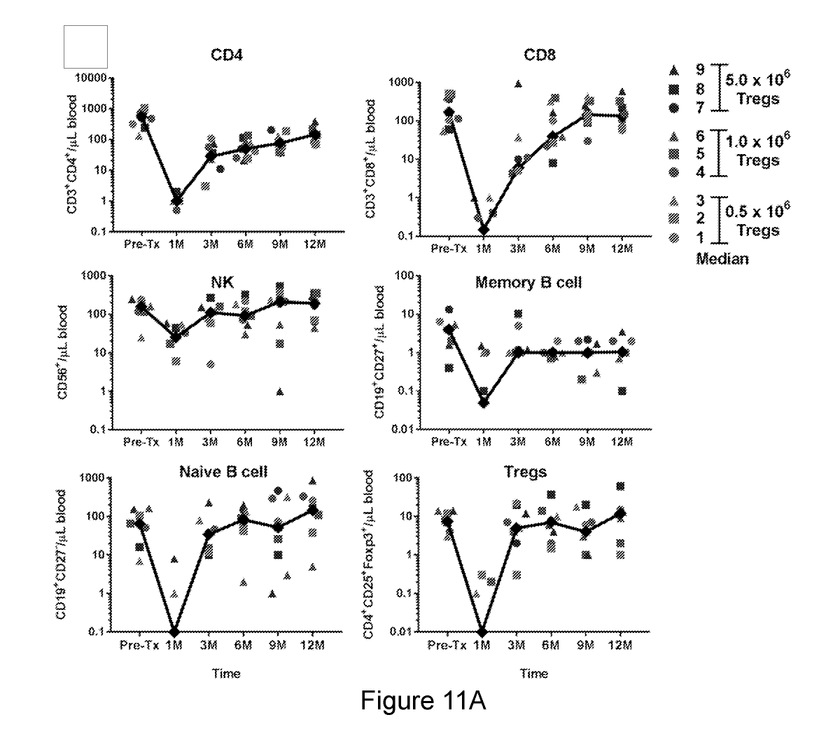

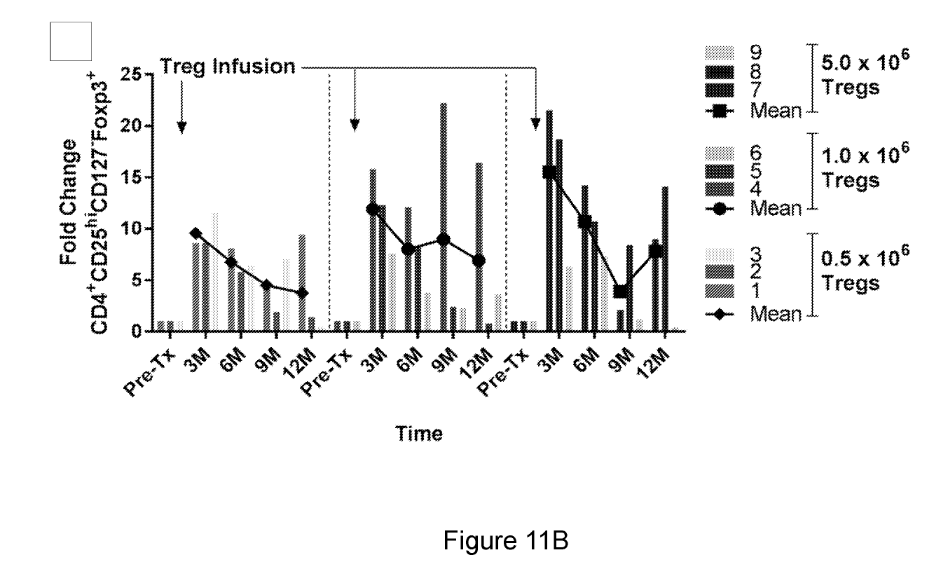

Kidney transplant recipients received alemtuzumab induction and this resulted in significant lymphodepletion (see FIG. 11A). The lymphodepletion was important for the later effectiveness of T-reg therapy. The subjects were begun on tacrolimus and mycophenolate-based immunosuppression and were converted from tacrolimus to sirolimus (rapamycin) at 30 days post-transplant (FIG. 6B) to provide an immunosuppressive milieu conducive to the survival of infused T-regs. By day +60 there was a recovery of the absolute numbers of NK cells, naïve B cells, T-regs (FIG. 11A) and CD14⁺ monocytes (not shown) prior to T-regs infusion which was performed on day +60 post-transplant. Most importantly, T-reg therapy resulted in 5-20 fold increase in the percentages of T-regs in all subjects and this increase remained stable in most patients until the end of the follow-up period of one year post-transplant (FIG. 11B).

Infusion of Expanded T-Regs into Kidney Transplant Recipients was Safe:

There were no serious adverse events attributable to the T-reg therapy in any subject (see Table 8). Protocol biopsies performed one month after T-reg therapy have not shown rejection (NR) and no donor specific antibody (DSA) development was observed at the time point. At 1 year post-transplant, there was a subclinical rejection (subject #8) with C4d deposition and DSA in one patient due to non-compliance with his immunosuppression and this was successfully treated.

TABLE 8

| | T-reg | Cell # | | | | | |
|---|---|---|---|---|---|---|---|
| | infusion | Admin- | | | | | |
| | (days post | istered | 3 m | 3 m | 1 yr | 1 yr | Graft |
| Subject # | transplant) | (10⁹) | Biopsy | DSA | Biopsy | DSA | Loss |
| 1 | 60 | 0.5 | NR | − | NR | + | NO |
| 2 | 60 | 0.5 | NR | − | NR | − | NO |
| 3 | 60 | 0.5 | NR | − | NR | − | NO |
| 4 | 60 | 1.0 | NR | − | NR | − | NO |
| 5 | 60 | 1.0 | NR | − | NR | − | NO |
| 6 | 60 | 1.0 | NR | − | NR | − | NO |
| 7 | 60 | 5.0 | NR | − | NR | − | NO |
| 8 | 60 | 5.0 | NR | − | SCR | + | NO |
| | | | | | C4d⁺ | | |
| 9 | 60 | 5.0 | NR | − | NR | − | NO |

At one year post-op low titer DSA was also observed in the only African American subject (#1) of the study (African Americans have historically been shown to be highly reactive to the donor). There have been no infectious complications attributable to T-reg infusion. Thus, the results from this phase I trial indicated that the foregoing T-reg therapy is safe.

Based upon these results, a dose of no less than 1×10E9 but no more than 5×10E9 cells for the T-reg therapy is expected to be efficacious and safe.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention. In some embodiment, percentages and amounts disclosed herein may vary in amount by ±10, 20, or 30%.

1. A method for selecting and expanding a population of CD4+/CD25+ T-regulatory cells, comprising:
(a) thawing a frozen apheresis sample received from an individual;
(b) selecting a population of CD4+/CD25+ T-regulatory (T-reg) cells from the thawed apheresis sample; and
(c) culturing the selected population of CD4+/CD25+ T-reg cells to produce an expanded population of CD4+/CD25+ T-reg cells,
wherein the expanded population of CD4+/CD25+ T-reg cells is larger than the selected population of CD4+/CD25+ T-reg cells by a factor of about 40.

2. The method claim 1, wherein a percentage of CD4+ T-reg cells in the expanded population of CD4+/CD25+ T-reg cells differs from a percentage of CD4+ cells in an expanded population of CD4+/CD25+ T-reg cells selected from a fresh, non-frozen apheresis product by less than about 3%.

3. The method claim 1, wherein a percentage of CD25+ T-reg cells in the expanded population of CD4+/CD25+ T-reg cells differs from a percentage of CD25+ cells in an expanded population of CD4+/CD25+ T-reg cells selected from a fresh, non-frozen apheresis product by less than about 5%.

4. A method for enriching and expanding CD4+/CD25+ T-regulatory (T-reg) cells from a cryopreserved apheresis sample, comprising:
(a) thawing the apheresis sample;
(b) suspending the thawed sample in a buffer comprising Human Serum Albumin (HSA), Magnesium Chloride (MgCl₂), and Dornase alfa;
(c) selecting a population of CD4+/CD25+ T-regulatory (T-reg) cells from the suspended apheresis sample to produce a selected population; and
(d) culturing the selected population of CD4+/CD25+ T-reg cells to produce an expanded population of CD4+/CD25+ T-reg cells,
wherein the expanded population of CD4+/CD25+ T-reg cells is larger than the selected population of CD4+/CD25+ T-reg cells by a factor of about 30.

5. A method for selecting CD25+ T-regulatory cells, comprising:
(a) thawing a cryopreserved apheresis product comprising T-cells;
(b) washing the thawed product in a buffer comprising Human Serum Albumin (HSA), Magnesium Chloride (MgCl₂), and Dornase alfa;
(c) incubating the thawed product with one or more capture surfaces comprising a binding agent for CD8+ and CD19+ cells;
(d) capturing the CD8+/CD19+ cells to the one or more surfaces;
(e) collecting a CD8/CD19 depleted product by washing the one or more surfaces with the buffer, and
(f) combing the CD8/CD19 depleted product with a capture surface for CD25+ cells,
(g) eluting cells captured on the capture surface for CD25+ cells with the buffer to provide a CD25+ enriched product;
wherein one or more of steps (c) though (g) use one or more buffers comprising HSA, MgCl₂, and Dornase alfa.

6. A method for expanding a CD25+ cell population, comprising:
(a) culturing CD25+ cells in a growth media supplemented with Interleukin-2 (IL-2), rapamycin, and Transforming Growth Factor Beta (TGF-β) in the presence of one or more surfaces comprising an anti-CD3+ antibody and anti-CD28+ antibody for about two days;
(b) adding IL-2 to the growth media and culturing the cells for about three days;
(c) adding additional growth media and IL-2, rapamycin, and TGF-β and culturing the cells for about two days;
(d) adding additional growth media and IL-2, rapamycin, TGF-β, and one or more surfaces comprising an anti-CD3+ antibody and anti-CD28+ antibody and culturing the cells for about two days;
(e) adding IL-2, rapamycin, and TGF-β, and culturing the cells for about 3 days;
(f) adding IL-2 and culturing the cells for about 2 days;
(g) adding additional growth media, IL-2, and TGF-β, and culturing the cells for about three days;
(h) adding IL-2 and culturing the cells for about two days; and
(i) separating the cell culture from the one or more capture surfaces to provide an expanded CD25+ cell population,
wherein no additional rapamycin is added to the cells beyond 9 days of culture.

7. A kit for providing an expanded and enriched CD4+/CD25+ T-reg cell population, comprising:
(a) a buffer comprising HSA, MgCl₂, and Dornase alfa; and
(b) instructions for use.

8. A kit for suppressing the immune system of an individual in need thereof, comprising:
(a) an expanded and enriched CD4+/CD25+ T-reg cell population; and
(b) instructions for use.

9. A composition, comprising:
(a) a buffer comprising HSA, MgCl₂, and Dornase alfa; and
(b) a thawed, previously cryopreserved, apheresis product comprising T-cells.

10. A composition comprising a previously frozen apheresis sample, a buffer comprising HSA, MgCl₂, and Dornase alfa.

11. A method for treating an organ transplant recipient, comprising administering to the recipient between about 1,000,000,000 and 5,000,000,000 CD4+/CD25+ T-reg cells selected and expanded from a frozen apheresis product.

12. The method of claim 11, wherein the frozen apheresis product was taken from the recipient prior to organ transplant.

13. The method of claim 11, wherein the frozen apheresis product was taken from a donor that is not the recipient.

14. The method of claim 11, wherein the method at least one of reduces, stops, and prevents a cellular immune response that causes cellular, organ, or tissue rejection in the recipient.

15. A method for treating an organ transplant recipient, comprising administering to the patient between about 1,000,000,000 and 5,000,000,000 CD4+/CD25+ T-reg cells from a population of cells prepared according claim 6.

16. A method for treating a patient suffering from an autoimmune disease, the method comprising administering to patient between about 1,000,000,000 and 5,000,000,000 CD4+/CD25+ T-reg cells selected and expanded from a frozen apheresis product.

17. A composition, comprising:
phosphate-buffered saline supplemented with 1 mM EDTA;
5% human serum albumin;
3.5 mM MgCl₂; and
50 U/mL Dornase alfa.

18. The composition of claim 17, further comprising a thawed, previously frozen, apheresis sample.
